(19) United States
(12) Patent Application Publication
(10) Pub. No.: US 2022/0364178 A1
(43) Pub. Date: Nov. 17, 2022

(54) URINARY RNA SIGNATURES IN RENAL CELL CARCINOMA (RCC)

(71) Applicants: University of Massachusetts, Boston, MA (US); Beth Israel Deaconess Medical Center, Inc., Boston, MA (US)

(72) Inventors: Jill A. Macoska, Quincy, MA (US); Justin Cotellessa, Dedham, MA (US); Manoj Bhasin, Johns Creek, GA (US)

(21) Appl. No.: 17/634,382

(22) PCT Filed: Aug. 13, 2020
(86) PCT No.: PCT/US2020/046206

§ 371 Date: Feb. 10, 2022

Related U.S. Application Data
(60) Provisional application No. 62/886,719, filed on Aug. 14, 2019.

(51) Int. Cl. C12Q 1/6886; G16H 20/10; G16H 20/40; G16H 50/20; G16H 50/30

(52) U.S. Cl. CPC C12Q 1/6886; G16H 20/10; G16H 20/40; G16H 50/20; G16H 50/30; C12Q 2600/158

(57) ABSTRACT

Methods of diagnosing, predicting risk of recurrence, and treating Renal Cell Carcinoma (RCC), e.g., clear cell Renal Cell Carcinoma (RCC).

### CLAIM OF PRIORITY

This application claims the benefit of U.S. Provisional Patent Application Ser. No. 62/886,719, filed on Aug. 14, 2019. The entire contents of the foregoing are hereby incorporated by reference.

### TECHNICAL FIELD

Described herein are methods of diagnosing, predicting risk of recurrence, and treating Renal Cell Carcinoma (RCC).

### BACKGROUND

Kidney Cancer will be newly diagnosed in 64,000, and will be the cause of death of 14,000 American men and women in 2017 (1). The death rate from renal cancer (20%) is higher than that for prostate (18%) or breast (16%) cancers, yet far fewer resources are available to improve the diagnosis and treatment of renal cancer (1). Surgical resection is the most common form of treatment for localized renal cancer.

### SUMMARY

Provided herein are methods for diagnosing renal cancer in a subject. In some embodiments, the methods include providing a sample comprising urine from a subject; treating the sample to remove cells (i.e., intact cells) from the urine; analyzing RNA present in the same to determine levels of MAX, MTIF3, RRP1, BUD31, and KLK2 transcripts in the sample; and diagnosing renal cancer in the subject based on the levels of MAX, MTIF3, RRP1, BUD31, and KLK2 in the sample.

Also provided herein are methods of providing a prognosis for a subject who has renal cancer. The methods include providing a sample comprising urine from a subject; treating the sample to remove cells (i.e., intact cells) from the urine; analyzing RNA present in the same to determine levels of MAX, MTIF3, RRP1, BUD31, and KLK2 transcripts in the sample; and determining a prognosis for the renal cancer in the subject based on the levels of MAX, MTIF3, RRP1, BUD31, and KLK2 in the sample.

Further, provided herein are methods for treating renal cancer in a subject or selecting a subject for treatment. The methods include providing a sample comprising urine from a subject; treating the sample to remove cells (i.e., intact cells) from the urine; analyzing RNA present in the same to determine levels of MAX, MTIF3, RRP1, BUD31, and KLK2 transcripts in the sample; and treating the renal cancer in the subject or selecting the subject based on the levels of MAX, MTIF3, RRP1, BUD31, and KLK2 in the sample.

In some embodiments, the methods include analyzing the sample to determine levels of one or more, e.g., all, of RHCG, EMP1, LOC102724761, SH3D19, and CDK14 and optionally one or more, e.g., all, of IFRD1, CEACAM1, SPINK5, PTCRA, and OR1C1, and optionally one or more, e.g., all, of S100A13, COQ6, AKAP7, BRDT, and ZNF578.

In some embodiments, the methods include calculating a score based on the levels of the transcripts in the sample; and diagnosing, determining a prognosis, or treating renal cancer based on the score.

In some embodiments, calculating a score comprises using a Random Forest Approach to discriminate between the probability that a urine sample is from a subject who has normal kidney tissue, non-recurrent kidney tumor, or recurrent kidney tumor, based on a molecular signature of the urine sample, e.g., a 5, 10, 15, or 20 transcript molecular signature.

In some embodiments, treating the renal cancer comprises one or more of surgical resection, radiofrequency or thermal ablation, radiation therapy, immunotherapy, and molecular-targeted therapy. In some embodiments, the immunotherapy comprises administration of one or more of Interferon (IFN) and interleukin-2 (IL-2); anti-programmed cell death-1 protein (PD-1) receptor antibodies; Bacillus Calmette-Guérin (BCG) vaccination; lymphokine-activated killer (LAK) cells with IL-2; tumor-infiltrating lymphocytes (TILs); lenalidamide; nonmyeloablative allogeneic peripheral blood stem-cell transplantation, and renal artery embolization. In some embodiments, the molecular targeted therapy comprises administration of one or more of sunitinib; lapatinib; pazopanib; temsirolimus; everolimus; bevacizumab (optionally in combination with interferon); lenvatinib (optionally in combination with everolimus); nivolumab; cabozantinib; sorafenib; and axitinib. In some embodiments, the chemotherapy comprises administration of one or more of Floxuridine (5-fluoro 2′-deoxyuridine [FUDR]), 5-fluorouracil (5-FU), vinblastine, paclitaxel (Taxol), carboplatin, ifosfamide, gemcitabine, and anthracycline (doxorubicin).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B. Histograms of the tumor stage and grade information for the 51 urine specimens used in RNASeq studies, stratified by grade (1A) and stage (1B) for recurrent (R, right hand bar in each pair) and non-recurrent (NR, left hand bar in each pair) tumors.

FIGS. 2A-2C. 2A. Principal Components Analysis of complete 77-transcript molecular signature. Stage (T) and grade (G) of tumors associated with urine specimens is indicated. Most of the urine samples segregate by recurrence status. 2B. Receiver-Operator Curve of complete and subset transcript molecular signatures; Area Under the Curve (AUA) is indicated for each signature. A 20-transcript subset demonstrated an AUC=0.939, while a 10-transcript set was AUC=0.921 and 5-transcript set was AUC=0.890. 2C. Genesets corresponding to the 5, 10, 15 and 20 transcript urinary molecular signatures.

FIG. 3. Principal Components Analysis of five GEO Dataset Reference Series demonstrates preferential expression of the 20-Transcript Urinary Molecular Signature in renal tumor (gray) compared to normal renal (black) tissues.

FIGS. 4A-4D. nanoString nCounter Evaluation of Urinary RNA Transcripts. 4A. Comparison of nanoString nCounter quantitation of unamplified endogenous (“housekeeping”) transcripts in 10 ng or 1 ng Cell Line RNA or Non-Patient, freshly collected urinary RNA. Urinary RNA levels are ˜10× lower than Cell Line levels. 4B. nanoString nCounter quantitation of the same transcripts in unamplified RNA purified from Emory Univ. archival (frozen) RCC recurrent patient urine. Urinary RNA levels are ˜½ that observed for Non-Patient, freshly collected urine. 4C. nanoString nCounter quantitation of the same transcripts in RNA purified from RCC recurrent Emory Univ. archival patient urine pre-amplified for 10 or 15 cycles (as indicated) with nested primers specific to the endogenous transcript probesets. Patient-derived transcript levels are ˜25×-higher at 10 cycles and ˜800× higher at 15 cycles pre-amplification compared to unamplified levels (as shown in B.). Cell line-derived transcript levels are ˜50×- and ˜500×-higher than those observed for 10 ng and 1 ng input RNA (as shown in 4A). 4D. FPKMs obtained from initial RNASeq analysis of DF/HCC RCC non-recurrent (NR) or recurrent (R) archival patient urine demonstrating relative concordance between nanoString nCounter- and RNASeq-measured transcript levels.

FIG. 5. Pre-Amplification Approach. Target-specific primers are used to pre-amplify cDNA made from Top 6 expressed housekeeping (RPL19, ACTB, GAPDH, RPLPO, LDHA, PGK1) transcripts or low-expressed CLTC urinary transcripts. All amplifications are in quadruplicate.

FIGS. 6A-6B. Random Forest Probability Plots predict normal kidney tissues, non-recurrent kidney tumors, or recurrent kidney tumors. 6A. Predicted RF scores of <0.5 indicate normal kidney tissue (white circles) scores of >0.5 indicate kidney tumor (black circles). 6B. Within tumors, scores of <0.75 indicate non-recurrent (NR) (white circles); scores of >0.75 indicate recurrent (R) tumors (black circles).

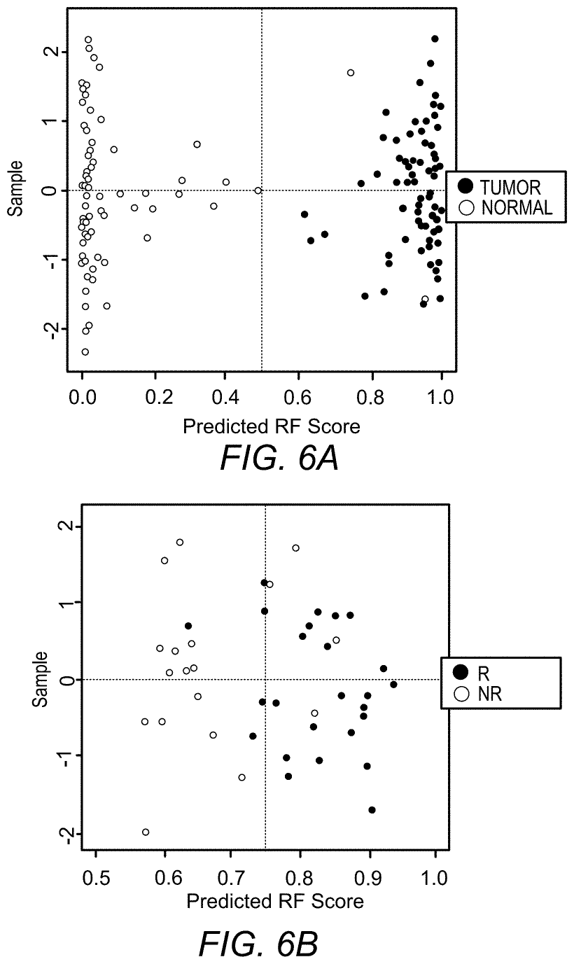

FIGS. 7A-7B. Exemplary illustrations of calculating a score as described herein.

### DETAILED DESCRIPTION

Few tools are available to help predict risk for recurrence in RCC. Data for nephrectomy in a large group of 255 patients showed that stratification by pathologic risk group can help predict cancer recurrence and progression for only a minority of patients [2]. Time to RCC recurrence is prognostic for cancer specific survival (CSS). A meta-analysis of a large comprehensive multi-center cohort of >13000 patients with initially localized RCC showed that time to recurrence ≤12 or ≤48 months post nephrectomy was associated with a CSS of 24% and 30%, respectively [3]. Another study reported that CSS for RCC patients whose tumors recurred ≤12 months post nephrectomy was only 23% [4]. Taken together, these studies suggest that RCC recurrence within 12 months of nephrectomy is associated with a greatly reduced CSS of <25%.

The relative risk for recurrence of other tumor types, notably breast cancers, can be assessed prior to surgical resection through the use of protein biomarkers applied to biopsy tissues. Information obtained from the examination of needle biopsy specimens for the presence or absence of the estrogen receptor, progesterone receptor, and HER2 proteins, combined with histopathological assessment of needle biopsy tissues, have proven highly informative for guiding the treatment of breast cancer patients that reduces risk for cancer recurrence [5]. However, this paradigm cannot be easily applied to prognostically assess kidney tumors. Both the National Comprehensive Cancer Network (NCCN) and European Association of Urology (EAU) Guidelines for Kidney Cancer recommend that CT of the abdomen and/or abdominal MRI are sufficient for the detection of renal masses, and that diagnostic percutaneous needle biopsy is recommended only for radiologically indeterminate renal masses [6, 7] [8]. Therefore, although multiple studies have reported the identification of tissue-based protein or RNA transcript biomarkers with potential utility for kidney cancer diagnosis and/or prognosis [9-12], these biomarkers cannot be utilized pre-nephrectomy in the absence of percutaneous needle biopsy.

The lack of routine assessment of percutaneous needle biopsies imposes limitations on the acquisition of prognostic information. However, these limitations could be addressed by the assessment of liquid biopsies for such prognostic information. Liquid biopsy specimens include saliva, serum, and urine. Of these, the biospecimen that most closely approximates the kidney both physically and metabolically is urine. Urine is commonly utilized as a rich source of information relevant to kidney function and kidney or bladder infection. With relatively recent advances in technology and bioinformatics tools, urine has also been found to be a good source of metabolic compounds [13, 14], proteins [14], microbes [15], and nucleic acids [16, 17, 18] that may inform and potentially stratify multiple disease states.

As demonstrated herein, small quantities of fresh or frozen (including archival samples stored >10 years) human urine specimens provide sufficient quantity and quality RNA for whole transcriptome sequencing. Moreover, application of this technology to urine specimens from 51 RCC patients and subjected to RNASeq, differential gene expression (DGE) and principal components (PC) analyses identified a distinct urinary transcript signatures present in urine at the time of nephrectomy. These signatures were able to distinguish between patients with non-recurrent or recurrent disease even better than tumor stage or grade.

These biomarkers can be applied pre-nephrectomy to predict risk for RCC recurrence within the critical 12-month post-nephrectomy period, and thereby identify patients at the time of resection that might benefit from closer surveillance, more extensive surgery, and/or immediate adjuvant therapy to improve RCC cancer-specific survival.

As noted above, the death rate from renal cancer (22%) is far higher than that for prostate (13%) or breast (17%) cancers, yet far fewer resources are available to improve the diagnosis and treatment of renal cancer [19]. Surgical resection is the most common form of treatment for renal cancer. Stratification by pathologic risk group can help predict cancer recurrence and progression, but for only for a minority of patients [2]. Unlike many other solid tumors, RCC diagnostic procedures do not typically include needle biopsy. Both the National Comprehensive Cancer Network (NCCN) and European Association of Urology (EAU) Guidelines for Kidney Cancer recommend that routine needle biopsy of renal masses is unnecessary as Computed Tomography (CT) of the abdomen and/or abdominal MRI are sufficient for the detection of renal masses, and patients undergo a curative-intent nephrectomy. Therefore, tumor needle biopsy tissues are not typically available for pre-surgical assessment of tumor risk for recurrence.

Methods of Diagnosis

Included herein are methods for diagnosing, e.g., determining presence or risk of recurrence of renal cell cancer (RCC), e.g., RCC, in a subject (e.g., a mammalian subject, e.g., a human or non-human mammal). The methods rely on detection of a plurality of transcripts (signature genes) as described herein, e.g., a signature comprising at least 5 genes, at least 10 genes, or at least 15 genes of the genes listed in Tables 2 and 3.

The methods include obtaining a sample comprising urine from a subject, and evaluating the presence and/or level of a set of transcripts as described herein in the sample, and comparing the presence and/or level with one or more references, e.g., a control reference that represents a normal level of the transcripts, e.g., a level in an unaffected subject who does not have RCC, or who had RCC but who has a low likelihood of recurrence, and/or a disease reference that represents a level of the transcripts associated with RCC, e.g., a level in a subject having RCC or a high likelihood of recurrence of RCC. In some embodiments, the methods include using an algorithm to calculate a score based on the levels of the transcripts, and the score is compared to a reference score that indicates whether the subject has RCC, or has a high likelihood of recurrence of RCC.

As used herein the term “sample”, when referring to the material to be tested for the presence of a biological marker using the method of the invention, includes urine and/or exosome or exosome-like microvesicles (U.S. Pat. No. 8,901,284) isolated from urine (20). In some embodiments, the urine samples are cell free, i.e., the whole urine is centrifuged, e.g., at 3,000 g for 10 minutes, the pellets discarded, and the supernatants used for the present methods.

In some embodiments, the subjects diagnosed using a method described herein have a risk of RCC or RCC that is higher than the general population, e.g., has one or more risk factors, e.g., genetic predisposition (e.g., von Hippel-Lindau syndrome, hereditary papillary renal carcinoma, Birt-Hogg-Dube syndrome, or hereditary renal carcinoma); or a presence or history of obesity, smoking, exposure to toxins (e.g., tricholoethylene), long-duration use of NSAIDs, use of phenacetin analgesics, long-term dialysis, renal transplant, hepatitis C, tuberous sclerosis, or kidney stones.

Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of transcripts described herein. Measurement of the level of a biomarker can be direct or indirect. For example, the abundance levels of the transcripts can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

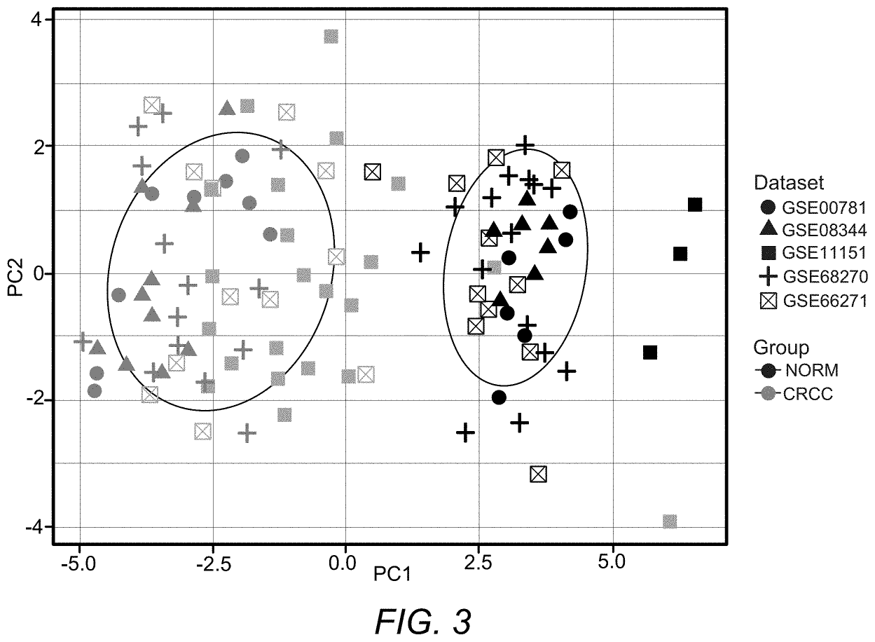

RT-PCR can be used to determine the expression profiles of biomarkers (see, e.g., U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such as RPLPO, ACTB, RPL19, PGK1, LDHA, GAPDH, CLTC (as shown in FIG. 4A) are most commonly used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

In some embodiments, the NANOSTRING NCOUNTER Platform digital color-coded barcode technology is used for direct multiplexed measurement of gene expression. The NanoString platform is FDA-approved for clinical diagnostics. NanoString currently markets the technically and clinically validated (ABCSG-8 Trial) Prosigna assay for use in postmenopausal women with hormone receptor-positive, node-negative (Stage I or II), or node-positive (Stage II) breast cancer. This assay, based on the PAM50 breast cancer genomic signature [21], has been shown to provide prognostic information beyond that obtained by the Clinical Treatment Score (CTS; derived from standard clinical covariates, including age, grade, tumor size, nodal status, and adjuvant therapy). In particular, the Prosigna assay successfully predicts the 10-year probability of distant recurrence among postmenopausal women with hormone receptor-positive or -negative breast cancer, and thereby identify patients who could benefit from adjuvant therapy.

In some embodiments, the presence and/or level of the transcripts, or the score determined based thereon, is comparable to the presence and/or level of the transcripts in a disease reference, and the subject has one or more symptoms associated with RCC, then the subject can be diagnosed with RCC. Symptoms of RCC can include flank pain, hematuria, and/or flank mass; less specific symptoms include weight loss, fever, hypertension, hypercalcemia, night sweats, malaise, or a (usually left) testicular varicocele in males. In some embodiments, the subject has no overt signs or symptoms of RCC, but the presence and/or level of one or more of the proteins evaluated is comparable to the presence and/or level of the protein(s) in the disease reference, then the subject has an increased risk of having or developing RCC, and can be subject to further evaluation for the presence of RCC, e.g., imaging or biopsy. In some embodiments, once it has been determined that a person has (or is likely to have) RCC, or has an increased risk of developing RCC, based on a method described herein, then further evaluation (e.g., using imaging methods) or a treatment, e.g., as known in the art or as described herein, can be administered. Imaging methods can include computed tomography (CT) or ultrasound, e.g., CT of the abdomen, preferably with pelvic CT; Magnetic resonance imaging (MRI), if venous involvement is suspected or the patient cannot tolerate contrast; Ultrasonography; Chest CT scan or chest x-ray; Excretory urography; Renal arteriography; Venography; Bone scan if bone metastasis is suspected or alkaline phosphatase level is elevated; and Brain CT or MRI if clinical manifestations suggest brain metastases.

Suitable reference values can be determined using methods known in the art, e.g., using standard clinical trial methodology and statistical analysis. The reference values can have any relevant form. In some cases, the reference comprises a predetermined value for a meaningful level of the transcripts or score, e.g., a control reference level or score that represents a normal level or score, e.g., a level or score in an unaffected subject or a subject who is not at risk of developing a disease described herein, and/or a disease reference that represents a level or score of the proteins associated with RCC, e.g., a level in a subject having RCC or a high likelihood of recurrence of RCC.

The predetermined level or score can be a single cut-off (threshold) value, such as a median or mean, or a level or score that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level or score is a level or score in the same subject, e.g., at a different time point, e.g., an earlier time point.

Subjects associated with predetermined values are typically referred to as reference subjects. For example, in some embodiments, a control reference subject does not have RCC, does not have recurrent RCC, or does not have a high likelihood of recurrence of RCC. In some cases it may be desirable that the control subject has had RCC but has not had a recurrence, and in other cases it may be desirable that a control subject does not have RCC.

A disease reference subject is one who has (or has an increased risk of developing) RCC or a recurrence of RCC. An increased risk is defined as a risk above the risk of subjects in the general population.

The predetermined value can depend upon the particular population of subjects (e.g., human subjects) selected. For example, an apparently healthy population will have a different ‘normal’ range of levels of transcripts or score than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

RCC Score

The present methods can include the use of an algorithm to calculate a score based on the expression levels of the signature gene transcripts. Thus, in some embodiments, the methods include applying an algorithm to expression levels for the transcripts (raw or normalized to an internal control) for MAX, MTIF3, RRP1, BUD31, and KLK2, and optionally one or more, e.g., all of RHCG, EMP1, LOC102724761, SH3D19, and CDK14 (e.g., a 10-gene signature), and optionally one or more, e.g., all, of IFRD1, CEACAM1, SPINK5, PTCRA, and OR1C1 (e.g., a 15-gene signature), and optionally one or more additional gene listed in Table 2. The methods can thus include calculating a score based on the levels of the transcripts in the sample; and diagnosing, determining a prognosis, or treating renal cancer based on the score

In some embodiments, the methods include determining levels of all of the following: BUD31 (BUD31 homolog), a spliceosomal protein important in cell tolerance to MYC hyper-activation, see Hsu et al., Nature. 2015 Sep. 17; 525(7569):384-8; MTIF3 (Mitochondrial Translational Initiation Factor 3), a translation initiation factor that is involved in mitochondrial protein synthesis; MAX (MYC Associated Factor X), a transcription factor that can heterodimerize with myc and is reportedly a tumor suppressor gene for renal oncocytomas (Korpershoek et al., The Journal of Clinical Endocrinology & Metabolism, 101(2):453-460 (2016); KLK2 (kallikrein related peptidase 2), which is thought to be involved in the carcinogenesis and tumor metastasis of prostate cancer (PCa) (Shang et al., Tumour Biol. 2014 March; 35(3):1881-90) but was reported not to be expressed in renal cell carcinoma in women (Clements et al., Clin Cancer Res. 1997 August; 3(8):1427-31); CDK14 (cyclin dependent kinase 14), a novel cyclin-dependent kinase reported to be overexpressed in a variety of cancers and related to their malignant behavior; IFRD1 (interferon related developmental regulator 1), a modifier gene for cystic fibrosis lung disease (Xu et al., Med Oncol. 2014 September; 31(9):135), with possible association between gene polymorphisms of IFRD1 and gastric cancer (Xu et al., Med Oncol. 2014 September; 31(9):135); CEACAM1 (Carcinoembryonic antigen-related cell adhesion molecules 1), de novo expression of which is found with progression of malignancy and metastatic spread in a number of cancers tissues including melanoma, Non-Small Cell Lung Carcinoma (NSCLC) as well as bladder, prostate, thyroid, breast, colon and gastric carcinomas (Fiori et al., Ann Ist Super Sanita. 2012; 48(2):161-71), but which is completely but reversibly downregulated in renal cell carcinoma (Kammerer et al., J. Pathol. 204(3): 258-267 (2004); RhCG (Rh type C-glycoprotein), the protein for which is reported to be downregulated in esophageal squamous cell carcinomas, but expressed in multiple squamous epithelia (Chen et al., Eur J Cancer. 2002 September; 38(14):1927-36), and expressed by chromophobe renal cell carcinoma and renal oncocytoma but not by clear cell renal cell carcinoma or by papillary renal cell carcinomas (Han et al., J Am Soc Nephrol. 2006 October; 17(10): 2670-2679.); EMP1 (epithelial membrane protein 1), which may play an important role as a negative regulator in breast cancer (Sun et al., Tumour Biol. 2014 April; 35(4):3347-54); SH3D19 (SH3 Domain Containing 19), expression of which correlated with AR expression in papillary RCC (pRCC) (Zhao et al., PLoS ONE 11(1): e0146505); SPINK5 (Serine Peptidase Inhibitor, Kazal Type 5), which is altered in larynx and hypopharynx tumors (Nair et al., Genes Cancer. 2015 July; 6(7-8): 328-340); RRP1 (Ribosomal RNA processing 1), which has been reported to be a host factor important for influenza A virus replication (Su et al., J. Virol. November 2015 vol. 89 no. 22 11245-11255); PTCRA (pre T-cell antigen receptor alpha), which is one of a set of multigene biomarkers for predicting sensitivity or resistance to an anti-cancer drug of interest, or multigene cancer prognostic biomarkers (see WO 2013095793 A1); LOC102724761, which is at present uncharacterized; and/or OR1C1 (olfactory receptor family 1 subfamily C member 1), which is one of 347 genes with high or low expression in Kidney renal clear cell carcinoma (see amp.pharm.mssm.edu/Harmonizome/gene_set/Kidney+renal+clear+cell+carcinoma KIRC TCGA-A3-3308-01A-02R-1325-07/TCGA+Signatures+of+Differentially+Expressed+Genes+for+Tumors.

In some embodiments, in addition to expression data, the algorithm can includes values representing one or more parameters relating to clinical status (e.g., TNM Tumor stage, Fuhrman Grade, and/or lymph node status; see, e.g., Klatte et al., World J Urol. 2018 December; 36(12):1943-1952), personal/lifestyle (age, gender, race, obesity/BMI, Hypertension, and/or Smoking); Carbonic anhydrase 9 (CA9) levels (see, e.g., Tostain et al., Eur J Cancer. 2010 December; 46(18):3141-8). In some embodiments age and body mass index (BMI) are analyzed as continuous variables, while gender, race, smoking history, hypertension, tumor stage (I vs. II vs. III) and Fuhrman grade (½ vs. ¾) status are categorical variables.

In some embodiments, the algorithm is a rank-based linear algorithm. A linear regression model useful in the methods described herein can include the variables (i.e., gene expression levels and other optional parameters) and coefficients, or weights, for combining expression levels. The coefficients can be calculated using a least-squares fit of the proposed model to a measure of risk of recurrence or presence of RCC.

In some embodiments, a decision trees based classifier based on a Random Forest Approach is used to discriminate between normal kidney tissues, non-recurrent kidney tumors and recurrent kidney tumors based on the urinary 20 transcript molecular signature. The classifier achieved an error rate (<5%) in predicting the chances of recurrence as well as depicted significant power in discriminating kidney tumors from normal kidney and discriminating recurrent from non-recurrent kidney tumors.

For example, in some embodiments the score is calculated as follows.

From the sequencing data for the 20 genes, X=x₁, . . . , xₙ with corresponding expression levels s Y=y₁, . . . , yₙ, bootstrap aggregating repeatedly (B times) selects a random sample with replacement (where an element may appear multiple times in the one sample) of the training set and fits decision trees to these samples:
For b=1, . . . , B:
1. Sample, with replacement, n training examples from X, Y; call these X_{b}, Y_{b}.
2. Train a classification or regression tree f_{b} on X_{b}, Y_{b}.
After training, predictions for unseen samples x′ can be made by averaging the predictions from all the individual regression trees on x′:

$$\hat{f} = \frac{1}{B} {\sum }_{b = 1}^{B} {f}_{b} ( {x}^{′} )$$

where \hat{f} is the probability that the urinary RNA signature predicts normal kidney (e.g., values <0.05), non-recurrent kidney tumors (e.g., values 0.05-<0.75) or recurrent kidney tumors (e.g., values 0.75-1.0).

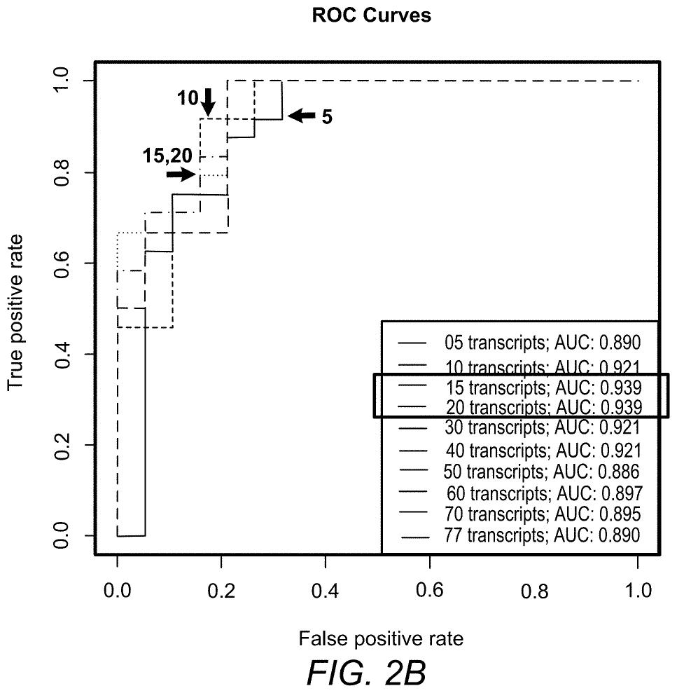

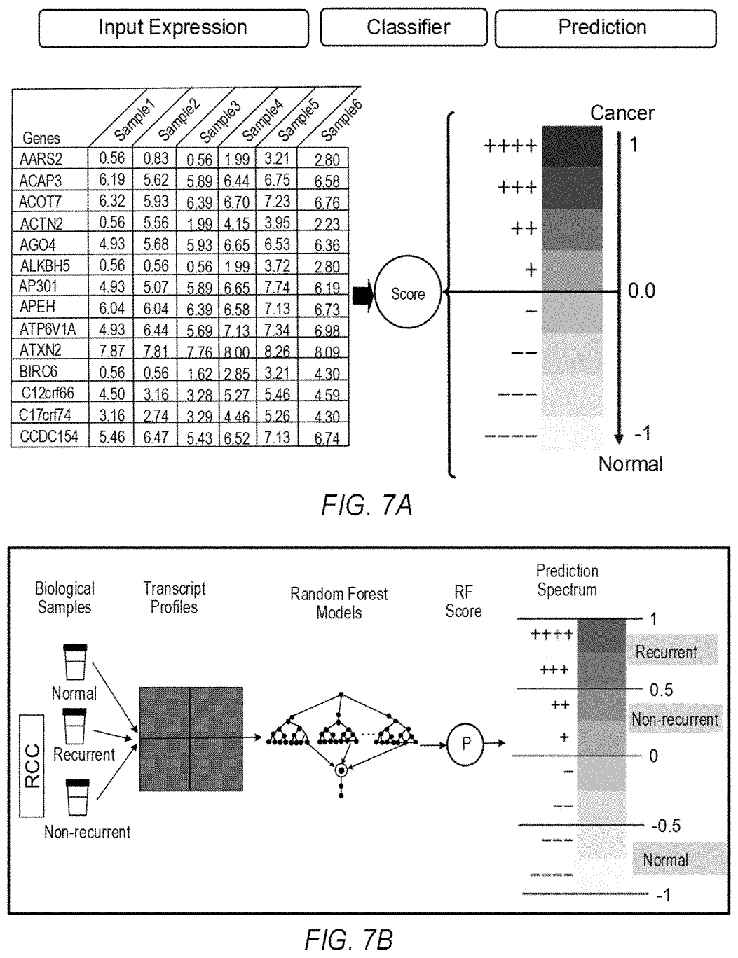

In some embodiments, the methods include determining the probability that the urinary RNA signature predicts normal kidney (values <0, or <0.05), non-recurrent kidney tumors (values 0.05-<0.75) or recurrent kidney tumors (values 0.75-1.0). FIG. 7A shows an exemplary method for calculating the score. In some embodiments, the mean (u) and standard deviation (sigma) of expression level is determined for all genes in the assay (e.g., the top 5, top 10, etc.) for all samples in the discovery set (see, e.g., FIG. 2C, or Table I) within one of two categories (recurrent or nonrecurrent) are used to calculate the z score based on the expression level (x) of any new urine specimen tested. Scores between 0-1 are indicative of disease or recurrent disease; those between −1-0 are indicative of no disease or non-recurrent disease (see FIG. 7A).

As shown in FIG. 7B, in some embodiments, normalized and batch effect corrected data is used for validation of random forest models developed on the basis of the 20 transcript Signature expression profile in the training set (e.g., as shown in FIG. 2C). Each sample can be given a random forest-based prediction score-based expression profile of the 20 transcripts signature. Then, samples with a positive RF score >0.5 will be predicted as recurrent samples, <0.5 will be predicted as non-recurrent, and <−0.5 will be predicted as non-cancerous or normals. The samples with borderline scores (near 0, e.g., within ±0.1 or 0.2) may not be classified RCC or non-cancerous to avoid misclassification errors in the first round of validation and performance calculation.

Methods of Treatment

The methods described herein include methods for the treatment of subjects diagnosed with RCC or with a high likelihood of recurrence of RCC based on the present methods. Generally, the methods include administering a therapeutically effective amount of a treatment as known in the art or described herein, to a subject who is in need of, or who has been determined to be in need of, such treatment.

As used in this context, to “treat” means to ameliorate at least one symptom of the disorder associated with RCC, e.g., to reduce the size, growth rate, likelihood of recurrence, or likelihood of metastases of the RCC.

Standard treatments include surgical resection (e.g., partial or total nephrectomy of primary tumors, and metastatic tumors), radiofrequency or thermal ablation (e.g., in subjects who cannot withstand surgery), radiation therapy (e.g., 4500 centigray (cGy) to 5500 cGy), immunotherapy, and molecular-targeted therapy.

Immune modulators can be used, including Interferon (IFN) and interleukin-2 (IL-2); anti-programmed cell death-1 protein (PD-1) receptor antibodies, e.g., nivolumab and similar agents; Bacillus Calmette-Guérin (BCG) vaccination; lymphokine-activated killer (LAK) cells with IL-2; tumor-infiltrating lymphocytes (TILs); lenalidamide; nonmyeloablative allogeneic peripheral blood stem-cell transplantation, and renal artery embolization (e.g., with ethanol and gelatin sponge pledgets).

Molecular targeted therapies can include sunitinib; lapatinib; pazopanib; temsirolimus; everolimus; bevacizumab, e.g., in combination with interferon; lenvatinib, e.g., in combination with everolimus; nivolumab; cabozantinib; sorafenib; and axitinib.

Chemotherapies can include Floxuridine (5-fluoro 2′-deoxyuridine [FUDR]), 5-fluorouracil (5-FU), vinblastine, paclitaxel (Taxol), carboplatin, ifosfamide, gemcitabine, and anthracycline (doxorubicin).

In subjects who have had surgical resection but who are determined to be at high risk of recurrence based upon a method described herein, the methods can include performing follow up, e.g., physical examination, comprehensive metabolic panel, and other laboratory tests as indicated, as well as imaging studies as described herein or known in the art, e.g., at least every 6 weeks, 8 weeks, 3 months, 4 months, or 6 months.

### EXAMPLES

The invention is further described in the following examples, which do not limit the scope of the invention described in the claims.

Methods

NanoString Assays for Urinary RNA. Briefly, urinary RNA was purified using the Qiagen miRNeasy Micro kit and checked for quality, quantity, and size distribution using the Agilent 2100 Bioanalyzer RNA 6000 Pico Chip. For the initial 66 urine sample Discovery Set, RNA recovery ranged from 0.17-51 total ng/sample with an average concentration of 9.1, and a median of 6.6, total ng/sample. The recovered RNA was degraded (average RIN=2.5), with fragments ranging in size from 20-500 nt and averaging 150 nt in length (Table 1).

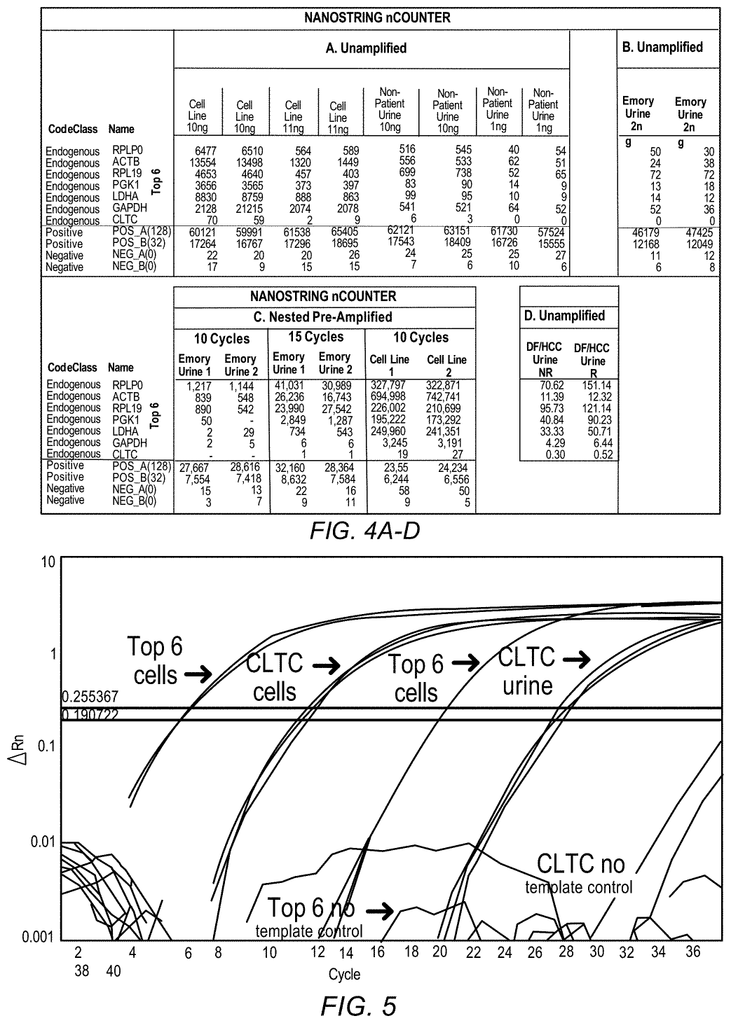

Due to the low total RNA recovery from urine, we utilized a pre-amplification approach prior to NanoString probeset annealing. The urinary RNA was subjected to first-strand cDNA synthesis using random hexamers. This step was followed by PCR amplification using target-specific primers (provided by NanoString) and tracked using Sybr green incorporation. As shown in FIG. 5, cell line cDNA amplified at lower cycles-to-threshold than urinary cDNA, as did the Top 6 expressed housekeeping transcripts (RPL19, ACTB, GAPDH, RPLPO, LDHA, PGK1) compared to the low-expressing CLTC transcript. Using this protocol we were able to: 1) Synthesize sufficient cDNA and pre-amplify sufficient template from 2 ng total urinary RNA to perform 2 NanoString assays comprising up to 800 unique barcoded probesets; and 2) detect patient-derived transcript levels from archival urine at levels 25×-higher at 10 cycles and 800×-higher at 15 cycles pre-amplification compared to unamplified levels (FIGS. 4B and 4C).

### Example 1. Clinical Need and Potential Utility of RCC Prognostic Biomarkers

Risk factors for RCC recurrence are mostly clinical and include tumor stage, regional lymph node status, tumor size, nuclear grade, and others (Leibovich et al., Cancer. 2003 Apr. 1; 97(7):1663-71). Host and tumor tissue-based gene and associated protein expression panels have been reported that predict RCC risk for recurrence, though these are not used in routine clinical practice [11, 20] (Rini et al., Lancet Oncol. 2015 June; 16(6):676-85; Schutz et al., Lancet Oncol. 2013 January; 14(1):81-7); The unavailability of routine diagnostic needle biopsies limit the utility of these panels to post-nephrectomy analysis. Because several lifestyle and epidemiological risk factors affect the incidence of RCC, it is possible that these factors have a role in RCC prognosis and recurrence [21]. We focused on common and pertinent risk factors including obesity, smoking and hypertension, race and family history.

Time to RCC recurrence is prognostic for cancer specific survival (CSS). A meta-analysis of a large comprehensive multi-center cohort of >13000 patients with initially localized RCC showed that recurrence ≤12 months or ≤48 post nephrectomy was associated with a CSS of 24% and 30%, respectively [3]. Another study reported that CSS for RCC patients whose tumors recurred ≤12 months post nephrectomy was only 23% [4]. Taken together, these studies suggest that RCC recurrence within 12 months of nephrectomy is associated with a greatly reduced CSS of <25%.

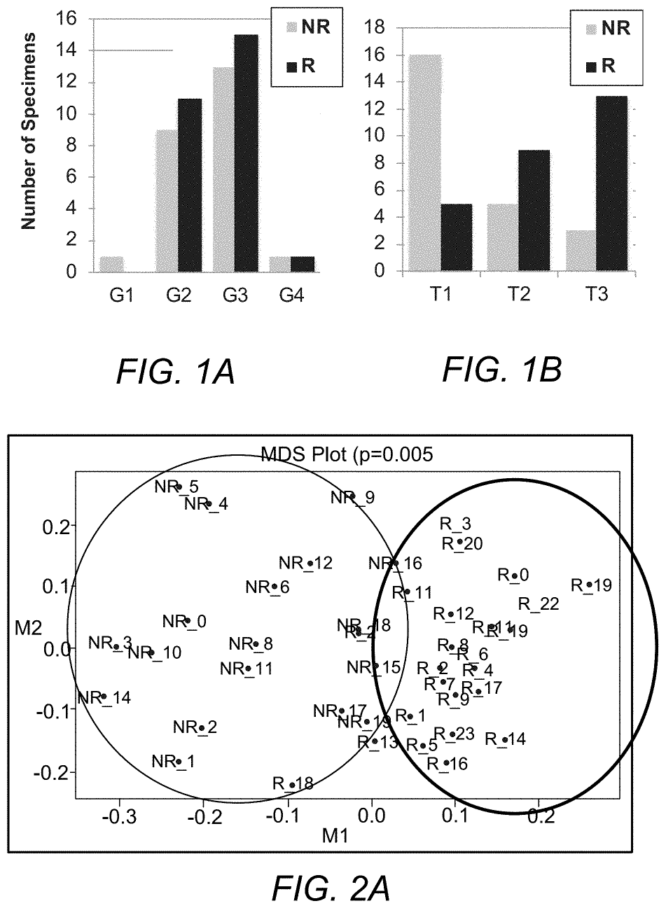

The utility of tumor stage and grade to predict tumor recurrence was assessed for the 51 patients included in our urinary transcript discovery set. Patients with metastatic RCC at presentation were excluded. As seen in Table 1, tumors from 24 non-recurrent and 27 recurrent RCC patients represented a wide spectrum of grade and stage disease. Within this group, tumor grade was not prognostic for disease recurrence and tumor stage was only marginally more aligned with disease outcome (FIG. 1B). Taken together with published findings [2-4], these data show that conventional pathological parameters do not provide adequate prognostic information to help guide post-nephrectomy management decisions, notably within the critical 12 month post-nephretomy period.

TABLE 1
Tumor stage and grade information for the
51 urine specimens used in RNASeq studies.

| | Non Recurrent | Recurrent |
|---|---|---|
| T1G1 | 1 | |
| T1G2 | 7 | |
| T1G3 | 7 | 4 |
| T1G4 | 1 | 1 |
| T2G1 | | |
| T2G2 | | 5 |
| T2G3 | 5 | 4 |
| T2G4 | | |
| T3G1 | | |
| T3G2 | 2 | 6 |
| T3G3 | 1 | 7 |
| T3G4 | | |
| Total | 24 | 27 |

### Example 2. Urine is a Rich Source of RNA Transcripts Useful for RCC Prognostic Biomarker Discovery and Validation

Human urine is a non-invasively collected ‘liquid biopsy’ biospecimen that is routinely used as a source of information for patient diagnosis and health monitoring. Rapid advances in technology have recently enabled the discovery of nucleic acids in urine that may have utility as biomarkers for disease status. The present studies used RNA-seq analysis to determine whether urine collected at the time of nephrectomy from RCC patients might harbor RNA transcripts (coding or non-coding) that could be identified using RNA sequencing; whether these transcripts might be differentially expressed in the urine of recurrent and non-recurrent RCC patients; and whether these transcripts could comprise an assay useful for the identification and validation of biomarkers predictive of risk for tumor recurrence in RCC patients.

The urine samples used in the RNASeq studies were cell free, i.e., the whole urine had been centrifuged, the pellets discarded, and the supernatants aliquoted, frozen, and inventoried as part of the biospecimen repository of the Dana-Farber Cancer Institute (DFCI) Kidney SPORE. Therefore, a protocol was developed to isolate RNA from cell-free rather than pelleted urine. Using a modification of the Qiagen miRNeasy Micro Kit, RNA was isolated first from freshly collected human urine and then from archival frozen human urine specimens. In both cases, the resulting RNA was degraded with fragments ranging in size from 20-500 nt and averaging 150 nt in length. 66 urine samples from the DF/HCC Kidney SPORE biorepository were initially subjected to RNA purification. Among these samples, the amount of RNA recovered ranged from 0.012-3.71 ng/ml, with an average recovery of 0.65 and median recovery of 0.30 ng/ul. The average RNA recovery within the recurrent and non-recurrent groups was similar (0.158 and 0.148 ng/ml, respectively). RNA Integrity Number (RIN) values were uniformly low and averaged 2.6. Based on RNA recovery, 51 of the 66 samples (24 from patients with non-recurrent disease and 28 from those with recurrent disease) were chosen to move forward to RNASeq analysis. It should be noted that, although it is likely that RNA recovered from these urine samples was exosomal in origin [22], no attempt was made to selectively purify exosomes prior to RNA recovery. The rationale for this was based on the desire to develop a urinary biomarker assay pipeline that did not require complex urine preparation or storage requirements that might impede later clinical implementation.

The RNASeq pipeline was developed to minimize loss of starting material (RNA) and to produce the highest number of reads and deepest coverage possible. With regard to the first requirement, ribodepletion, which would have resulted in loss of starting material, was not performed because: 5S, 16S and 28S rRNA peaks were not observed on the Agilent bioanalyzer traces; an initial MiSeq study (see below) revealed minimal rRNA content in urinary RNA; and rRNA has not been reported as part of exosomal RNA (the likely origin of urinary RNA).

The construction of the sequencing libraries was also optimized. For library construction, RNA should be ligated to adapters, then reverse-transcribed to cDNA. Adapter ligation requires intact 5′ phosphate and 3′ OH groups. Initial studies using freshly collected urine suggested poor adapter ligation. However, when the RNA was first end-repaired using T4 polynucleotide kinase (PNK), adapter ligation was greatly improved. Therefore, RNA recovered from the RCC archival urine samples was end-repaired with PNK prior to adapter ligation and reverse transcription. Initial library preparations from RNA samples from 2 freshly collected and 2 RCC archival specimens were first tested on the Illumina MiSeq. These studies showed that the urine libraries possessed an extremely high GC content that interfered with detection and quantitation of AT content. Therefore, an additional PhiX Control library (Illumina) was included in the subsequent HiSeq studies. For RNASeq of urinary transcript libraries, paired end sequencing libraries (including 3 batch controls prepared from freshly-collected non-RCC urine) were amplified for 21 cycles (due to low input) and 2 nM per library were clustered and sequenced on the Illumina HiSeq 2500. The Q30 for the run was 93.8%, indicating fewer than 1 in 1000 base calls were predicted to be incorrect), and the average reads per sample was 22.4 million.

### Example 3. Identification of a 15-Transcript Urinary Molecular Signature Associated with Disease Status

BAM files were trimmed and converted to fastq, then passed through the Bowtie/TopHat/Cufflinks/CummeRbund (Tuxedo) suite of analysis programs. Mapping rates averaged 85%. Differential gene expression (DGE) between RNAs isolated from the urine of recurrent and non-recurrent RCC patients was determined using Cuffdiff2. These analyses identified 77 highly significant (p<0.005 and q<0.0025) differentially expressed transcripts (Table 2). Of these, 44 were coding transcripts and the remaining 34 comprised 23 miRNAs, 2 snoRNAs, or 8 uncharacterized LOC-designated transcripts. Principal components analysis (FIG. 2A) revealed that most of the urine samples segregated according to tumor recurrence status. Interestingly, the molecular signature successfully identified low stage/low grade tumors that recurred as well as high stage/high grade tumors that did not. Receiver-Operator Curve (ROC) (FIG. 2B) analysis revealed an AUC=0.89 for the complete 77-transcript molecular signature, which improved using a smaller 15 subset transcript signature (AUC=0.939) and a 10-transcript signature (AUC 0.921); a 5-transcript signature provided substantial information as well (AUC 0.890) (Tables 2 and 3, and FIG. 2C). Therefore, these discovery studies successfully identified a urinary RNA molecular signature that could distinguish between non-recurrent and recurrent RCC and provide the rationale for the combinatorial and validation studies proposed in this application.

TABLE 2
Urinary transcripts associated with RCC recurrence

| IDENTIFIER | NR | R | NR | R | log2_fold_change | test_stat | p_value | q_value | Significant |
|---|---|---|---|---|---|---|---|---|---|
| BUD31 | NR | R | 4.5292 | 37.2548 | 3.0401 | 3.8998 | 0.0001 | 0.0042 | yes |
| MTIF3 | NR | R | 12.9263 | 92.5012 | 2.8392 | 10.7695 | 0.0001 | 0.0025 | yes |
| MAX | NR | R | 1.7551 | 8.8618 | 2.3360 | 0.9962 | 0.0001 | 0.0025 | yes |
| KLK2 | NR | R | 31.9494 | 142.8230 | 2.1604 | 2.0673 | 0.0001 | 0.0025 | yes |
| CDK14 | NR | R | 2.8560 | 10.2034 | 1.8370 | 2.9778 | 0.0001 | 0.0025 | yes |
| IFRD1 | NR | R | 38.4681 | 7.9093 | −2.2821 | −7.9919 | 0.0001 | 0.0042 | yes |
| CEACAM1 | NR | R | 42.2499 | 5.8524 | −2.8519 | −8.5067 | 0.0001 | 0.0025 | yes |
| RHCG | NR | R | 6.0462 | 0.7705 | −2.9722 | −1.1289 | 0.0001 | 0.0025 | yes |
| EMP1 | NR | R | 353.2110 | 39.2705 | −3.1690 | −9.9292 | 0.0001 | 0.0025 | yes |
| SH3D19 | NR | R | 62.9080 | 6.9547 | −3.1772 | −1.3921 | 0.0001 | 0.0025 | yes |
| SPINK5 | NR | R | 2121.2100 | 224.1250 | −3.2425 | −50.5154 | 0.0001 | 0.0025 | yes |
| RRP1 | NR | R | 9.7773 | 0.8751 | −3.4819 | −0.4783 | 0.0001 | 0.0025 | yes |
| PTCRA | NR | R | 47.8037 | 2.7296 | −4.1304 | −1.1028 | 0.0001 | 0.0025 | yes |
| LOC102724761 | NR | R | 1183.0700 | 44.9504 | −4.7181 | −29.6335 | 0.0001 | 0.0025 | yes |
| OR1C1 | NR | R | 121.3900 | 4.3244 | −4.8110 | −13.6598 | 0.0001 | 0.0025 | yes |
| LOC285593 | NR | R | 339.2470 | 3627.5500 | 3.4186 | 31.4122 | 0.0001 | 0.0025 | yes |
| AKAP7 | NR | R | 35.0650 | 365.3210 | 3.3811 | 114.3220 | 0.0001 | 0.0025 | yes |
| ZNF773 | NR | R | 7.2400 | 69.9356 | 3.2720 | 2.0317 | 0.0001 | 0.0042 | yes |
| COQ6 | NR | R | 2.5823 | 20.5128 | 2.9898 | 3.4946 | 0.0001 | 0.0025 | yes |
| S100A13 | NR | R | 2.1659 | 15.8149 | 2.8683 | 2.0101 | 0.0001 | 0.0025 | yes |
| RSRC1 | NR | R | 21.5631 | 98.8161 | 2.1962 | 20.3484 | 0.0001 | 0.0042 | yes |
| IRAK4 | NR | R | 66.2351 | 205.0160 | 1.6301 | 18.0806 | 0.0001 | 0.0025 | yes |
| CARD8 | NR | R | 59.0393 | 23.5348 | −1.3269 | −5.7267 | 0.0001 | 0.0025 | yes |
| TCP11L2 | NR | R | 188.4830 | 60.8654 | −1.6307 | −19.0476 | 0.0001 | 0.0042 | yes |
| ANXA2 | NR | R | 75.7325 | 23.0712 | −1.7148 | −2.5859 | 0.0001 | 0.0042 | yes |
| TRDN | NR | R | 276.3890 | 76.7506 | −1.8485 | −26.5200 | 0.0001 | 0.0025 | yes |
| SLC11A2 | NR | R | 237.0330 | 64.3968 | −1.8800 | −5.7251 | 0.0001 | 0.0042 | yes |
| MUC1 | NR | R | 40.9986 | 10.0950 | −2.0219 | −1.7137 | 0.0001 | 0.0025 | yes |
| PDCD10 | NR | R | 869.4570 | 168.3450 | −2.3687 | −62.8861 | 0.0001 | 0.0042 | yes |
| BRDT | NR | R | 359.7990 | 55.7238 | −2.6908 | −11.3028 | 0.0001 | 0.0025 | yes |
| LOC102724054 | NR | R | 222.3910 | 30.2966 | −2.8759 | −7.7514 | 0.0001 | 0.0042 | yes |
| FAM213B | NR | R | 8.3842 | 1.1414 | −2.8769 | −0.5667 | 0.0001 | 0.0025 | yes |
| IL8 | NR | R | 12987.3000 | 1729.3300 | −2.9088 | −84.5118 | 0.0001 | 0.0025 | yes |
| CBLB | NR | R | 357.3510 | 40.5867 | −3.1383 | −1.6054 | 0.0001 | 0.0025 | yes |
| SNX10 | NR | R | 313.1000 | 34.3292 | −3.1891 | −40.9007 | 0.0001 | 0.0025 | yes |
| ADCY10 | NR | R | 192.6550 | 18.6679 | −3.3674 | −29.2728 | 0.0001 | 0.0025 | yes |
| ALOX12 | NR | R | 47.6015 | 4.2471 | −3.4865 | −2.4368 | 0.0001 | 0.0025 | yes |
| NCF2 | NR | R | 54.2232 | 4.8201 | −3.4918 | −4.7977 | 0.0001 | 0.0025 | yes |
| ZNF578 | NR | R | 250.9840 | 13.2609 | −4.2423 | −16.1173 | 0.0001 | 0.0025 | yes |
| LCE3D | NR | R | 16.3769 | 0.6969 | −4.5546 | −0.6889 | 0.0001 | 0.0025 | yes |
| NBPF25P | NR | R | 1131.6400 | 44.6099 | −4.6649 | −11.6867 | 0.0001 | 0.0025 | yes |
| LOC102723807 | NR | R | 37.4933 | 0.6112 | −5.9388 | −0.5437 | 0.0001 | 0.0025 | yes |
| LOC727710 | NR | R | 15.0174 | 0.2115 | −6.1498 | −0.6084 | 0.0001 | 0.0025 | yes |
| LOC102724045 | NR | R | 547.7790 | 6.5578 | −6.3842 | −5.0836 | 0.0001 | 0.0042 | yes |
| OR56A3 | NR | R | 35.0835 | 0.0630 | −9.1214 | −1.5524 | 0.0001 | 0.0025 | yes |
| SNORD3C | NR | R | 0.0000 | 22.5581 | CC | CC | 0.0001 | 0.0025 | yes |
| TRNAC15 | NR | R | 2.6574 | 0.0000 | CC | CC | 0.0001 | 0.0042 | yes |
| TRNAC18 | NR | R | 5.7460 | 0.0000 | CC | CC | 0.0001 | 0.0025 | yes |
| TRNAY8 | NR | R | 8.2164 | 0.0000 | CC | CC | 0.0001 | 0.0025 | yes |
| PTH | NR | R | 65.4717 | 0.0000 | CC | CC | 0.0001 | 0.0025 | yes |
| SNORD116-20 | NR | R | 521.4710 | 0.0000 | CC | CC | 0.0001 | 0.0025 | yes |
| LOC102724008 | NR | R | 2191.5900 | 0.0000 | CC | CC | 0.0001 | 0.0025 | yes |
| LOC102724495 | NR | R | 3507.6500 | 0.0000 | CC | CC | 0.0001 | 0.0025 | yes |
| MIR3175 | NR | R | 0.0000 | 9.9098 | CC | CC | 0.0001 | 0.0025 | yes |
| MIR4723 | NR | R | 0.0000 | 38.8319 | CC | CC | 0.0001 | 0.0025 | yes |
| MIR4745 | NR | R | 0.0000 | 10.4983 | CC | CC | 0.0001 | 0.0025 | yes |
| MIR6800 | NR | R | 0.0000 | 6.9348 | CC | CC | 0.0001 | 0.0025 | yes |
| MIR6802 | NR | R | 0.0000 | 49.8199 | CC | CC | 0.0001 | 0.0025 | yes |
| MIR877 | NR | R | 0.8021 | 15.9980 | 4.3179 | 0.1405 | 0.0001 | 0.0025 | yes |
| MIR4726 | NR | R | 1.5630 | 19.3359 | 3.6289 | 0.2144 | 0.0001 | 0.0025 | yes |
| MIR338 | NR | R | 1.9124 | 23.7844 | 3.6365 | 0.2880 | 0.0001 | 0.0025 | yes |
| MIR6722 | NR | R | 9.7988 | 0.2214 | −5.4677 | −0.0504 | 0.0001 | 0.0025 | yes |
| MIR548E | NR | R | 13.9101 | 0.0000 | CC | CC | 0.0001 | 0.0025 | yes |
| MIR3065 | NR | R | 14.4319 | 0.0000 | CC | CC | 0.0001 | 0.0025 | yes |
| MIR7844 | NR | R | 16.2146 | 0.0000 | CC | CC | 0.0001 | 0.0025 | yes |
| MIR6825 | NR | R | 17.6623 | 0.1194 | −7.2083 | −0.0377 | 0.0001 | 0.0025 | yes |
| MIR3064 | NR | R | 24.8303 | 1.5251 | −4.0252 | −0.2555 | 0.0001 | 0.0025 | yes |
| MIR566 | NR | R | 25.6998 | 0.0000 | CC | CC | 0.0001 | 0.0025 | yes |
| MIR885 | NR | R | 28.7111 | 0.0000 | CC | CC | 0.0001 | 0.0025 | yes |
| MIR6875 | NR | R | 33.5093 | 0.0000 | CC | CC | 0.0001 | 0.0025 | yes |
| MIR598 | NR | R | 51.8407 | 0.0000 | CC | CC | 0.0001 | 0.0025 | yes |
| MIR4733 | NR | R | 59.2997 | 0.0000 | CC | CC | 0.0001 | 0.0025 | yes |
| MIR6773 | NR | R | 86.0404 | 4.5385 | −4.2447 | −0.8813 | 0.0001 | 0.0025 | yes |
| MIR561 | NR | R | 139.1880 | 0.9226 | −7.2372 | −0.3138 | 0.0001 | 0.0025 | yes |
| MIR4676 | NR | R | 166.5050 | 2743.8200 | 4.0425 | 7.8440 | 0.0001 | 0.0025 | yes |
| MIR3129 | NR | R | 273.5160 | 0.0000 | CC | CC | 0.0001 | 0.0025 | yes |
| MIRLET7F2 | NR | R | 486.9910 | 21.6787 | −4.4895 | −1.5933 | 0.0001 | 0.0025 | yes |

Transcript levels are in Fragments Per Kilobase of transcript per Million (FPKM).
CC = cannot calculate; R = recurrent; NR = Non-Recurrent.

TABLE 3
Genes in 5 and 10-gene signatures

| 5-Genes | 10-Genes |
|---|---|
| MAX | MAX |
| MTIF3 | MTIF3 |
| RRP1 | RRP1 |
| BUD31 | BUD31 |
| KLK2 | KLK2 |
| | RHCG |
| | EMP1 |
| | LOC102724761 |
| | SH3D19 |
| | CDK14 |

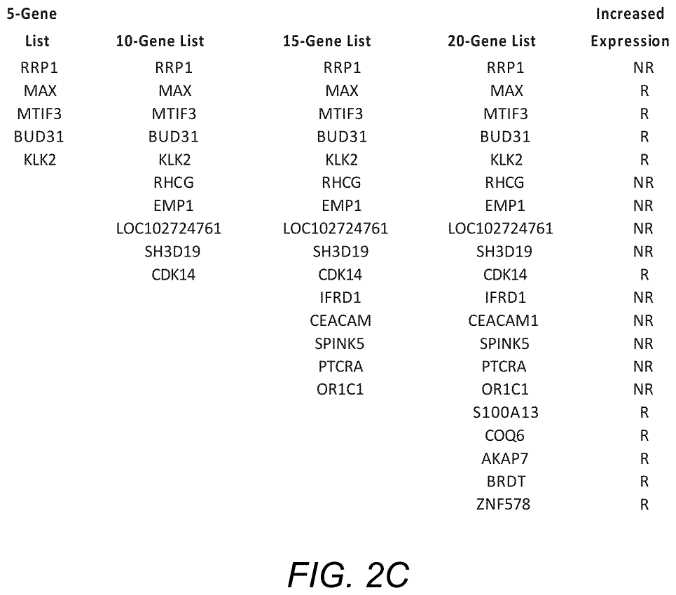

Principle component analysis of the expression pattern of the 15-Transcript Urinary Molecular Signature in five Gene Expression Omnibus (GEO) DataSet Reference Panels showed that it was preferentially expressed in RNA derived from malignant rather than normal kidney tissue (FIG. 3). This finding lends confidence to that the signature is clearly kidney tumor-derived.

### Example 4. NanoString nCounter Assay Validated RNASeq Urinary RNA Transcript Levels

Preliminary validation studies were conducted to assess the correlation between urinary transcript levels for endogenous housekeeping genes and controls detected by RNASeq and NanoString nCounter platforms. As seen in FIG. 4A) Comparison of NanoString nCounter quantitation of unamplified endogenous (“housekeeping”) transcripts in 10 ng or 1 ng Cell Line RNA or Non-Patient, freshly collected urinary RNA. Urinary RNA levels are ˜10× lower than Cell Line levels; 4B) NanoString nCounter quantitation of the same transcripts in unamplified RNA purified from Emory Univ. archival (frozen) RCC recurrent patient urine. Urinary RNA levels are +½ that observed for Non-Patient, freshly collected urine; 4C) NanoString nCounter quantitation of the same transcripts in RNA purified from RCC recurrent Emory Univ. archival patient urine pre-amplified for 10 or 15 cycles (as indicated) with nested primers specific to the endogenous transcript probesets. Patient-derived transcript levels are ˜25×-higher at 10 cycles and ˜800× higher at 15 cycles pre-amplification compared to unamplified levels (as shown in 4B). Cell line-derived transcript levels are ˜50×- and 500×-higher than those observed for 10 ng and 1 ng input RNA (as shown in 4A); 4D) FPKMs obtained from initial RNASeq analysis of DF/HCC RCC non-recurrent (NR) or recurrent (R) archival patient urine demonstrating relative concordance between NanoString nCounter- and RNASeq-measured transcript levels. Moreover, this data shows that the most informative transcripts are very highly up-regulated in the urine from patients with recurrent disease (Table 2), hence, clearly amenable to NanoString nCounter detection.

### REFERENCES

1. Siegel R L, Miller K D, Jemal A. Cancer statistics, 2017. C A Cancer J Clin. 2017; 67(1):7-30.
2. Gabr A H, Gdor Y, Strope S A, Roberts W W, Wolf J S, Jr. Patient and pathologic correlates with perioperative and long-term outcomes of laparoscopic radical nephrectomy. Urology. 2009; 74(3):635-40.
3. Brookman-May S D, May M, Shariat S F, Novara G, Zigeuner R, Cindolo L, et al. Time to recurrence is a significant predictor of cancer-specific survival after recurrence in patients with recurrent renal cell carcinoma—results from a comprehensive multi-centre database (CORONA/SATURN-Project). BJU international. 2013; 112(7):909-16.
4. Rodriguez-Covarrubias F, Gomez-Alvarado M O, Sotomayor M, Castillejos-Molina R, Mendez-Probst C E, Gabilondo F, et al. Time to recurrence after nephrectomy as a predictor of cancer-specific survival in localized clear-cell renal cell carcinoma. Urol Int. 2011; 86(1):47-52.
5. Gradishar W, Salerno K E. NCCN Guidelines Update: Breast Cancer. J Natl Compr Cam Netw. 2016; 14(5 Suppl):641-4.
6. Motzer R J, Agarwal N, Beard C, Bolger G B, Boston B, Carducci M A, et al. NCCN clinical practice guidelines in oncology: kidney cancer. J Natl Compr Canc Netw. 2009; 7(6):618-30.
7. New NCCN Guidelines Include Evidence Blocks to Illustrate Value in Breast, Colon, Kidney, and Rectal Cancers [Internet]. 2016 [cited March]. Available from: ncbi.nlm.nih.gov/pubmed/27396028
8. Ljungberg B, Bensalah K, Canfield S, Dabestani S, Hofmann F, Hora M, et al. EAU guidelines on renal cell carcinoma: 2014 update. European urology. 2015; 67(5):913-24.
9. Zacchia M, Vilasi A, Capasso A, Morelli F, De Vita F, Capasso G. Genomic and proteomic approaches to renal cell carcinoma. J Nephrol. 2011; 24(2):155-64.
10. Rydzanicz M, Wrzesinski T, Bluyssen H A, Wesoly J. Genomics and epigenomics of clear cell renal cell carcinoma: recent developments and potential applications. Cancer Lett. 2013; 341(2):111-26.
11. Brooks S A, Brannon A R, Parker J S, Fisher J C, Sen O, Kattan M W, et al. ClearCode34: A prognostic risk predictor for localized clear cell renal cell carcinoma. European urology. 2014; 66(1):77-84.
12. Brannon A R, Reddy A, Seiler M, Arreola A, Moore D T, Pruthi R S, et al. Molecular Stratification of Clear Cell Renal Cell Carcinoma by Consensus Clustering Reveals Distinct Subtypes and Survival Patterns. Genes Cancer. 2010; 1(2):152-63.
13. Hao L, Greer T, Page D, Shi Y, Vezina C M, Macoska J A, et al. In-Depth Characterization and Validation of Human Urine Metabolomes Reveal Novel Metabolic Signatures of Lower Urinary Tract Symptoms. Sci Rep. 2016; 6:30869.
14. Di Meo A, Pasic M D, Yousef G M. Proteomics and peptidomics: moving toward precision medicine in urological malignancies. Oncotarget. 2016.
15. Whiteside S A, Razvi H, Dave S, Reid G, Burton J P. The microbiome of the urinary tract—a role beyond infection. Nature reviews Urology. 2015; 12(2):81-90.
16. Chen S, Zhao J, Cui L, Liu Y. Urinary circulating DNA detection for dynamic tracking of EGFR mutations for NSCLC patients treated with EGFR-TKIs. Clin Transl Oncol. 2017 March; 19(3):332-340.
17. Salvi S, Gurioli G, Martignano F, Foca F, Gunelli R, Cicchetti G, De Giorgi U, Zoli W, Calistri D, Casadio V. Urine Cell-Free DNA Integrity Analysis for Early Detection of Prostate Cancer Patients. Dis Markers. 2015; 2015:574120.
18. Parker J S, Mullins M, Cheang M C, Leung S, Voduc D, Vickery T, et al. Supervised risk predictor of breast cancer based on intrinsic subtypes. J Clin Oncol. 2009; 27(8):1160-7.
19. Cho E, Adami H O, Lindblad P. Epidemiology of renal cell cancer. Hematol Oncol Clin North Am. 2011; 25(4):651-65.
20. Khurana R, Ranches G, Schafferer S, Lukasser M, Rudnicki M, Mayer G, Hüttenhofer A. Identification of urinary exosomal noncoding RNAs as novel biomarkers in chronic kidney disease. RNA. 2017 February; 23(2):142-152.
21. Nielsen T, Wallden B, Schaper C, Ferree S, Liu S, Gao D, Barry G, Dowidar N, Maysuria M, Storhoff J. Analytical validation of the PAM50-based Prosigna Breast Cancer Prognostic Gene Signature Assay and nCounter Analysis System using formalin-fixed paraffin-embedded breast tumor specimens. BMC Cancer. 2014; 14:177. Epub 2014/03/15.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
providing a sample comprising urine from a subject;
treating the sample to remove cells from the urine;
analyzing RNA present in the same to determine levels of MAX, MTIF3, RRP1, BUD31, and KLK2 transcripts in the sample.

2. (canceled)

3. A method of treating renal cancer in a subject or selecting a subject for treatment, the method comprising:
providing a sample comprising urine from a subject;
treating the sample to remove cells from the urine;
analyzing RNA present in the same to determine levels of MAX, MTIF3, RRP1, BUD31, and KLK2 transcripts in the sample; and
treating the renal cancer in the subject or selecting the subject based on the levels of MAX, MTIF3, RRP1, BUD31, and KLK2 in the sample.

4. The method of claim 1, further comprising analyzing the sample to determine levels of one or more of RHCG, EMP1, LOC102724761, SH3D19, and CDK14, and optionally one or more all, of IFRD1, CEACAM1, SPINK5, PTCRA, and OR1C1, and optionally one or more, e.g., all, of S100A13, COQ6, AKAP7, BRDT, and ZNF578.

5. The method of claim 1, further comprising: calculating a score based on the levels of the transcripts in the sample.

6. The method of claim 5, wherein calculating a score comprises using a Random Forest Approach to discriminate between the probability that a sample is normal kidney tissue, non-recurrent kidney tumor or recurrent kidney tumor based on the levels of MAX, MTIF3, RRP1, BUD31, and KLK2.

7. The method of claim 3, wherein treating the renal cancer comprises one or more of surgical resection, radiofrequency or thermal ablation, radiation therapy, immunotherapy, chemotherapy, and molecular-targeted therapy.

8. The method of claim 7, wherein the immunotherapy comprises administration of one or more of Interferon (IFN) and interleukin-2 (IL-2); anti-programmed cell death-1 protein (PD-1) receptor antibodies; Bacillus Calmette-Guérin (BCG) vaccination; lymphokine-activated killer (LAK) cells with IL-2; tumor-infiltrating lymphocytes (TILs); lenalidamide; nonmyeloablative allogeneic peripheral blood stem-cell transplantation, and renal artery embolization.

9. The method of claim 7, wherein the molecular targeted therapy comprises administration of one or more of sunitinib; lapatinib; pazopanib; temsirolimus; everolimus; bevacizumab (optionally in combination with interferon); lenvatinib (optionally in combination with everolimus); nivolumab; cabozantinib; sorafenib; and axitinib.

10. The method of claim 7, wherein the chemotherapy comprises administration of one or more of Floxuridine (5-fluoro 2′-deoxyuridine [FUDR]), 5-fluorouracil (5-FU), vinblastine, paclitaxel (Taxol), carboplatin, ifosfamide, gemcitabine, and anthracycline (doxorubicin).

11. The method of claim 3, further comprising analyzing the sample to determine levels of one or more of RHCG, EMP1, LOC102724761, SH3D19, and CDK14, and optionally one or more of IFRD1, CEACAM1, SPINK5, PTCRA, and OR1C1, and optionally one or more of S100A13, COQ6, AKAP7, BRDT, and ZNF578.

12. The method of claim 3, further comprising:
calculating a score based on the levels of the transcripts in the sample; and
treating the renal cancer based on the score.

13. The method of claim 12, wherein calculating a score comprises using a Random Forest Approach to discriminate between the probability that a sample is normal kidney tissue, non-recurrent kidney tumor or recurrent kidney tumor based on the levels of MAX, MTIF3, RRP1, BUD31, and KLK2.

14. The method of claim 5, wherein the method comprises determining levels of transcripts of MAX, MTIF3, RRP1, BUD31, KLK2, RHCG, EMP1, LOC102724761, SH3D19, CDK14, IFRD1, CEACAM1, SPINK5, PTCRA, OR1C1, S100A13, COQ6, AKAP7, BRDT, and ZNF578, and calculating a score based on the levels of the transcripts.

15. The method of claim 14, comprising using a Random Forest Approach to discriminate between the probability that a sample is normal kidney tissue, non-recurrent kidney tumor or recurrent kidney tumor based on the levels of the transcripts.

16. The method of claim 12, wherein the method comprises determining levels of transcripts of MAX, MTIF3, RRP1, BUD31, KLK2, RHCG, EMP1, LOC102724761, SH3D19, CDK14, IFRD1, CEACAM1, SPINK5, PTCRA, OR1C1, S100A13, COQ6, AKAP7, BRDT, and ZNF578, and calculating a score based on the levels of the transcripts.

17. The method of claim 16, comprising using a Random Forest Approach to discriminate between the probability that a sample is normal kidney tissue, non-recurrent kidney tumor or recurrent kidney tumor based on the levels of the transcripts.
